(19) United States
(12) Patent Application Publication
(10) Pub. No.: US 2006/0272367 A1
(43) Pub. Date: Dec. 7, 2006

(54) Attachable restraining device

(76) Inventor: Matt W. Kriesel, Melrose, WI (US)

Correspondence Address: Steven L. Schmid, 44 Coral Street, Atlantic Beach, FL, 32233, US

(21) Appl. No.: 11/324,771

(22) Filed: Jan. 3, 2006

Related U.S. Application Data
(60) Provisional application No. 60/641,653, filed on Jan. 4, 2005.

(51) Int. Cl. E05B 75/00; A61B 19/00

(52) U.S. Cl. 70/16; 128/869; 70/18

(57) ABSTRACT

Disclosed is a law enforcement strap restraint having a flexible strap forming two separate loops that may be tightened by a cross sectional member attached near the end tips of the strap. The cross sectional member serves as push bar device for moving the strap and tightening the loops to entrap a person's wrists. The device further includes an elongated member attached to the strap at one end and at the other end is selectively removable attachment device for attaching the device to a relatively stationary object. The cross sectional member is slidedably connected to the elongated member and is guided by the elongated member when the cross sectional member is engaged.

### RELATED APPLICATIONS

This application relies upon U.S. Provisional Patent Application Ser. No. 60/641653 filed Jan. 4, 2005.

### FIELD OF THE INVENTION

The present invention relates to law enforcement restraining devices and more particularly relates to a two-loop strap restraint having an extended member and attachment device for attaching the restraint to a stationary object.

### BACKGROUND

Various types and constructions of flexible material wrist restraints for use by law enforcement personnel in dealing with subduing suspects and controlling prisoners are known. Such restraints in most instances are made of a polymeric based material and embody strap parts which can be formed into loops for girding both wrists of a person, and means to secure or lock the loops, the locking means commonly being teeth or projections carried on the strap parts and a cooperating latch or stop usually carried elsewhere on the strap parts for engaging the teeth or projections to effect a stop function which maintains the loops secured.

Representative of the mentioned constructions are U.S. Pat. Nos. 4,071,023; 4,909,051; 4,910,831; 5,088,158 and 5,159,728. These patents disclose using generally fiat strap parts as the loop forming component. U.S. Pat. No. 4,854,138 discloses using a flexible braid or rope-like element to form the ropes.

A limitation of certain of the representative constructions is that the strap formed loops while securable adequately around a person's wrists leave little or no part of the device extending in any significant manner from the loops where such conveniently, surely and safely could be used as a person control element by an officer moving the restrained person to a vehicle or wanting to put the person down on the ground. With these constructions, generally the officer must place his hands on the device proper and proximal those of the restrained person who could grab the officer and start a struggle. Where a part of the device extends from the loops, such is either inadequate from the point of good grabbing length or not suited to providing a secure hold.

U.S. Pat. No. 4,854,138 discloses a restraining device using a flexible braid to form loops which when tightened with a lock bar results in there being a length of the braid remaining which is said to provide a lead for leading the restrained person. This lead, however, is a flaccid, not easily held element that in a struggle likely can be pulled from an officer's hands. Further, the lead is of two side by side braid lengths which due to the nature of the material from which made and especially after making an overhand knot therein as taught in the patent, will almost always lay close to each other, and it would be problematical to rely on how quickly and effectively an officer could spread the two lengths to obtain a more firm grip than the simple grasping the two lengths together for effecting a control.

It also is to be noted with regard to the '138 patent device, that if a person whose wrists have been restrained with closely wrist fitting loops becomes unruly and further suppressive action thereby indicated, such could not be practiced by temporary imposition of additional loop tightening to produce person compliance since once a loop size is set with the device it cannot be altered to loosen what would be a significant continuous painful and circulation endangering loop tightness.

Accordingly, it is desirable that a wrist restraining device be provided which not only securely holds a person's wrists but also embodies a means for restraining an individual without having the need to come in contact with the individual.

### SUMMARY

The law enforcement strap restraint embodying the present invention does not require that a law enforcement office come in direct contact with a prisoner. Additionally, there is no need for a key to unlock either loop once it has been applied around the wrist of a restrained prisoner since the strap is formed from a of polymeric material. Furthermore the strap is disposable once it is cut away and removed.

In greater detail, the law enforcement strap restraint includes a flexible strap forming two separate loops. The flexible strap includes two ends that are pulled though a locking mechanism to decrease the size of the formed loops. A cross sectional member attached near the ends of the strap, whereby the cross sectional member tightens the loops by pulling the strap ends though the locking mechanisms to entrap a person's wrists. An elongated member is included having a first and second end, the first end of the elongated member is attached to the strap and the second end comprises a selectively removable attachment device for attaching the law enforcement strap device to an object. The cross sectional member is slidedably connected to the elongated member whereby the cross sectional member is guided by the elongated member when the cross sectional member is engaged.

Among the advantages of the lightweight strap restraint are those resulting from the fact that the law enforcement officer does not come in direct contact with the prisoner in the application of the restraint. An additionally, one can easily carry a plurality of them. These stiffly flexible strap restraints can readily be looped around each other in numerous ways for being coupled together with one or more of the loops being attached to the same or other individuals or animals, and such coupling may include anchoring one of the loops to a stationary member such as a post, pipe, fence or other object or to an anchoring member in a transport vehicle. The restraining device referred to herein will not require a key or other unlatching element and is inexpensive and disposable in that once it has been applied, it is removed by simply cutting or snipping it from the person being restrained.

### DRAWINGS

In the Drawings:

FIG. 1 depicts a cross sectional view of the restraint device.

### DETAILED DESCRIPTION

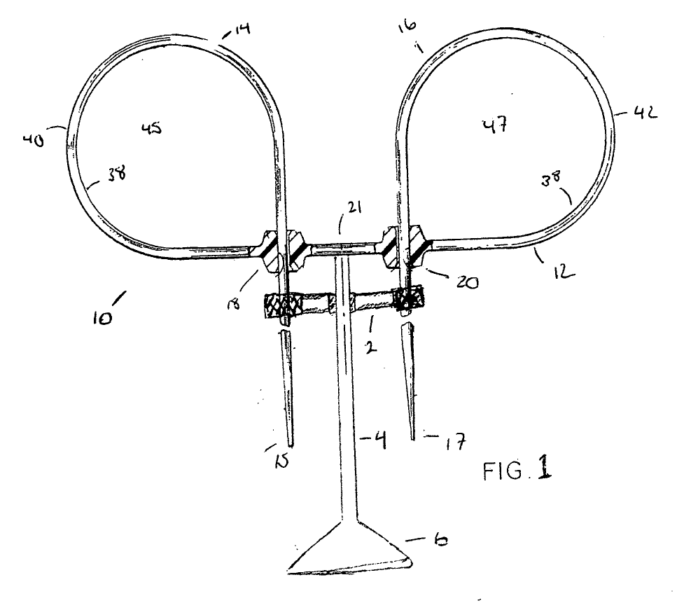

Referring to FIG. 1 a law enforcement strap restraint embodying the present invention is referred to generally by the reference numeral 10 and comprises a single, elongated, integral, stiffly flexible strap 12 having first and second ratchet portions 14 and 16, respectively, with respective rounded tips 15 and 17. A cross sectional member 2 is attached near the rounded tips 15 and 17. The cross sectional member serves as push bar device for moving the strap and tightening the loops 45 and 47 to entrap a person's wrists. Additionally, an elongated member 4 is attached to the strap 12. The cross sectional member 2 is slidedably connected to the elongated member 4 and is guided by the elongated member 4 when the cross sectional member 2 is engaged. Furthermore, the elongated member 4 has attached at one end an attachment device 6. The attachment device 6 may be a section cup or other means for selectively attaching the law enforcement strap restraint 10 to a stationary object.

The law enforcement strap 10 may be molded of a lightweight, strong, tough, stiffly flexible, high-strength, engineering-type, thermoplastic, polymeric material, for example, it is molded of engineering grade Nylon 66 or Nylon 6 each being a virgin resin impact-modified to enhance flexibility and having a tensile strength of at least about 8,000 psi (ASTM Test Method D 638) and a flexural strength of at least about 13,500 psi. A cross sectional member 2 is attached neat the rounded tips 15 and 17.

The strap 12 can be cut with a pair of large cutting snips or large cutting shears, but such cutting is not easily done. The strap 12 contains first and second socket bosses 18 and 20, respectively, which are centrally located in the strap 12 generally midway between the first and second rounded tips 15 and 17. It is to be noted that the restraint 10 is symmetrical about a transverse centerline 19 and thus the socket bosses 18 and 20 are each located the same relatively small distance on either side of a centerline. There is a relatively wide bridging portion 21 of the strap 12 which spans between and is integral with the respective socket bosses and is reinforced by two longitudinal ribs 54 for strongly and securely interconnecting these sockets. This restraint 10 is also advantageously symmetrical about a longitudinal centerline axis so that it is conveniently reversible end-for-end when being applied. In other words, the user need not waste time in checking which tip 15 and 17 is to be inserted first, because of the advantageous symmetry.

The first and second spaced socket bosses 18 and 20 have central openings extending there through which openings are substantially perpendicular to the plane of the elongated straight strap 12. The socket 18 includes a downwardly extending resilient latch pawl that is inclined downwardly in a direction away from a transverse centerline. This latch pawl projects into the opening of the socket 18 for ratchet latching purposes explained hereinafter. The other socket 20 also includes an identical downwardly inclined resilient latch pawl which projects downwardly into the opening in a direction away from the centerline. These latch pawls are molded of the same polymeric material used for molding the strap 12 and socket bosses 18 and 20, so that these pawls are integral with their respective socket bosses.

The strap 12 includes a first narrowed tip pull portion extending inwardly from the rounded tip 15 to the ratchet portion 14. This narrowed tip pull portion may have a width in the range of about 50% to about 70% of the width of the ratchet portion 14, for example being about 58%. This tip pull is tapered and includes a plurality of small ridges extending transversely with respect to a longitudinal centerline. These small ridges have a saw-tooth configuration with their abrupt faces being on the mesial surface of each ridge and with their sloping faces being on the distal surface. The purpose of this saw-toothed tip pull is to provide a high frictional grip by an officer when pulling on it after the narrowed tip pull portion has been inserted fully through the socket opening for enabling firm manual pulling of the ratchet portion 14 through the socket 18. The narrowed tip pull leads to a ratchet toothed channel surface having larger saw-tooth ridges , which slope in the same direction as the smaller ridges of the tip pull. Thus, these larger saw-tooth ridges have abrupt mesial faces and sloping distal faces for providing a stiffly flexible longitudinally extending first ratchet portion 14 of the strap 12.

The ratchet portion 14 has a broad trough-shaped (broad U-shaped) configuration forming a channel in which is located the saw-toothed surface. The broad flat surfaces 38 of the first and second ratchet portions 14 and 16 of the strap 12 are considered the “bottom” or “inward” surfaces, because such surfaces 38 are intended to face inwardly toward the wrist of the restrained person. Conversely, the surface containing the channel is considered the “top” or “outward” surface. This top surface must face the resilient latch pawl for it to engage against the abrupt mesial ridge faces for latching the ratchet portion 14 in the socket.

The second ratchet portion of the strap 12 is molded in the same shape as the first ratchet portion 14, except that this second ratchet portion is the symmetrical mirror image of the first. There is a second narrowed tip pull portion which is molded in the same shape as the first tip pull portion except that this second narrowed tip pull is the symmetrical mirror image of the first.

In operation of the law enforcement strap 10, the strap may function by having an individual place their wrists within the formed loops 45 and 47 of the strap. Once the wrists are placed within the loops 45 and 47, the loops may be ratcheted up or tightened to securely entrap the wrists in the loops 45 and 47. The loops 45 and 47 are ratcheted up by engaging the cross sectional member 2 by sliding the member 2 down or along the elongated member. Since the cross sectional member 2 is securely attached to the strap ends 15 and 17 the loops 45 and 47 become smaller.

In greater detail, the first ratchet portion 14 is flexed with its bottom (inward) surface 38 facing the wrist or ankle or other object being encircled to form a loop with its narrowed tip pull portion 29 having been inserted through the socket 18 in order for the stiffly flexible ratchet portion to be pulled there through to form a first loop 45. Then, the second tip pull 30 and ratchet portion 16 of the strap 12 are looped in the opposite direction and inserted into and pulled through the socket 20 to form a second loop 47.

The relatively close spacing of the socket bosses 18 and 20 serves to restrain the person whose wrists are in the respective loop configurations 45 and 47. Moreover, since the ratchet portions 14 and 16 are stiffly flexible, they can be flexed as may be desired or required for configuring into various shapes and sizes of loops for snugly and accurately conforming to the perimeter of the encircled ankle, wrist or other anchoring object. In addition, there can be the same or different sizes of loops. The looping arrangement is simple and the flexing direction for each ratchet portion 14 and 16 is inwardly toward the transverse centerline 19. Thus, insertion of the tip pull portions 29 and 30 into the sockets followed by the snugging of loops 45 and 47 is easily done by the person to be restrained by engaging the cross sectional member 2.

While Applicants have set forth embodiments as illustrated and described above, it is recognized that variations may be made with respect to disclosed embodiments. Therefore, while the invention has been disclosed in various forms only, it will be obvious to those skilled in the art that many additions, deletions and modifications can be made without departing from the spirit and scope of this invention, and no undue limits should be imposed except as set forth in the following claims.

1. A law enforcement strap restraint comprising:
a flexible strap forming two separate loops and the flexible having a first and second end;
a cross sectional member attached near the ends of the strap, whereby the cross sectional member tightens the loops to entrap a person's wrists; and
an elongated member having a first and second end, the first end of the elongated member is attached to the strap and the second end comprises a selectively removable attachment device for attaching the law enforcement strap restraint to an object, the cross sectional member is slidedably connected to the elongated member whereby the cross sectional member is guided by the elongated member when the cross sectional member is engaged.

2. The law enforcement strap restraint of claim 1, wherein the flexible straps are comprised of a polymeric material.

3. The law enforcement strap restraint of claim 1, wherein the cross sectional member includes a ratcheting member for tightening the loops to entrap a person's wrists.

4. The law enforcement strap restraint of claim 1, wherein the selectively removable attachment device comprises a section cup.

5. The law enforcement strap restraint of claim 1, wherein the selectively removable attachment device includes an adhesive.

6. The law enforcement strap restraint of claim 5, wherein the adhesive includes a release paper.

7. The law enforcement strap restraint of claim 5, wherein the adhesive is releasable.

8. The law enforcement strap restraint of claim 1, wherein the cross sectional member includes clasp for tightening the loops to entrap a person's wrists.

9. The law enforcement strap restraint of claim 1, wherein the selectively removable attachment device includes an magnet.

10. The law enforcement strap restraint of claim 1, wherein the selectively removable attachment device includes a hook and loop device.
